(19) United States
(12) Patent Application Publication
(10) Pub. No.: US 2024/0373975 A1
(43) Pub. Date: Nov. 14, 2024

(54) METHOD OF PROVIDING FOOT ORTHOTICS

(71) Applicant: CUSTOM FOOT INSERTS PTY LTD, New South Wales (AU)

(72) Inventors: Benjamin HINTON, New South Wales (AU); William WOOD, New South Wales (AU); Lina WOOD, New South Wales (AU)

(73) Assignee: CUSTOM FOOT INSERTS PTY LTD, New South Wales (AU)

(21) Appl. No.: 18/574,486

(22) PCT Filed: Jul. 7, 2022
(86) PCT No.: PCT/AU2022/050712

§ 371 Date: Dec. 27, 2023

(30) Foreign Application Priority Data
Jul. 7, 2021 (AU) 2021902066

(51) Int. Cl. A43B 7/1415; G06T 7/00

(52) U.S. Cl. CPC A43B 7/1415; G06T 7/0012; G06T 2207/30004

(57) ABSTRACT

Providing a foot orthotic (10) to a person including presenting a map of a foot and leg divided into predetermined regions to the person, receiving a selection from the person of one or more regions from the map corresponding to one or more locations where the person is experiencing pain or discomfort: defining a plurality of foot orthotic types. each foot orthotic type adapted to address pain or discomfort in the one or more regions where the person is experiencing pain or discomfort. and dispensing an orthotic (10) from the plurality of orthotic types such that the defined orthotic (10) is adapted to address pain or discomfort in the region where the person first experienced the pain or discomfort.

### FIELD OF THE INVENTION

The present invention relates to foot orthotics and, in particular, to a method of prescribing and providing same.

The invention has been developed primarily in respect of a method of prescribing a foot orthotic to assist in treatment of foot and leg pain and will be described hereinafter with reference to this application. However, it will be appreciated that the invention is not limited to this particular field of use and is applicable to assist in treatment of foot and leg misalignment, for example.

### BACKGROUND OF THE INVENTION

A foot orthotic, or orthosis in the singular, is an apparatus that is disposed intermediate the sole of the foot and a shoe inner surface that is used to apply a force or support to the foot in an upward direction. Foot orthotics are produced by qualified health practitioners termed orthotists and are shaped and sized to correct foot alignment or deformity or to relieve for minimise pain and discomfort in a person by redistributing the load of the foot.

Foot orthotics can be produced from a cast or a scan of the foot. This can be done by producing an orthotic completely or by modifying a preformed template to properly fit the foot of a patient to provide the support required. Here, a trained practitioner creates the cast or scans the foot of a patient and then may modify this to produce a final orthotic, or they can modify a preformed orthotic templated so as to provide the desired shape to support the foot of the patient correctly.

This process requires specialist skill depending on the correction required which the practitioner calculates and producers. There are many conditions that may require the use of an orthotic to support a foot to reduce pain or to correct the stance of the patient. Such conditions requiring corrective orthotic to include pronated or supinated feet and flat footedness (pes planus) which typically require manipulation by a skilled practitioner to arrive at the correct orthotic shape to provide the appropriate support for the foot. A well-known technique for use in aligning a foot to produce an orthotic such as to correct a pronation can be found in WO01/19246 (Smith) which describes apparatus used in aligning a foot to be able to produce a corrected orthotic.

The foot orthotists also address issues of pain in a person caused by their feet or lower legs and prescribe orthotics to correct this in some circumstances, for example pain in part of a foot or tendon, et cetera. In such cases, skilled input from a practitioner is necessary and a patient will be referred to a podiatrist or other practitioner from medical and paramedical practitioners which identify that a foot orthotic is or may be required.

Unfortunately, this can be a relatively time-consuming and expensive procedure to correct what might be minor pain in a foot or lower leg of a patient. This gives rise to preformed or prefabricated generically sized orthotics that are available to consumers without consideration by a podiatrist or orthosist. Typically, however, these prefabricated orthotics suffer from the disadvantage that fit of a similar shoe size are in fact always the same physical size or shape and can have parts of the foot in different spots relative to other parts of the foot between patients. For example, the location or pitch of the calcaneus bone at the heel of the foot or the specific size of a medial longitudinal arch which varies in people with the same sized foot.

### OBJECT OF THE INVENTION

The object of the invention is a desire to provide a method of prescribing and providing foot orthotics to a person in need thereof that minimises or eliminates skilled practitioner input, or to provide a useful alternative.

### SUMMARY OF THE INVENTION

According to a first aspect of the present invention there is disclosed a method of providing a foot orthotic to a person in need thereof, the method including the steps of: presenting a map of a foot and leg divided into predetermined regions to the person; receiving a selection from the person of one or more regions from the map corresponding to one or more locations where the person is experiencing pain or discomfort; defining a plurality of foot orthotic types, each foot orthotic type adapted to address pain or discomfort in the one or more regions where the person is experiencing pain or discomfort; dispensing an orthotic from the plurality of orthotic types such that the defined orthotic is adapted to address pain or discomfort in the region where the person first experienced the pain or discomfort.

It can be seen that there is advantageously provided a method of producing an dispensing a foot orthotic to a person in need without engaging a specialist practitioner. Further, the orthotics can be produced to correspond to the specific size and shape of a person who can simply scan their foot and provide an electronic 3-D image thereof so that a standard foot orthotic blank can be modified during production. Furthermore, the defined types of orthotics can be useful to a person across a very wide range of areas in which they are experiencing pain or discomfort and a standardised process is provided.

### BRIEF DESCRIPTION OF THE DRAWINGS

A preferred embodiment of the invention will now be described, by way of example only, with reference to the accompanying drawings in which:

FIG. 1 is a diagrammatic questionnaire identifying areas of pain in a person;

FIG. 2 is this a tabular listing of areas of first pain and corresponding orthotics to be dispensed in response to an area/s of pain;

FIG. 3 shows examples of each of the orthotic types to be dispensed; and

FIG. 4 shows a schematic process diagram for the production of a defined orthotic according to the preferred embodiment.

### DETAILED DESCRIPTION

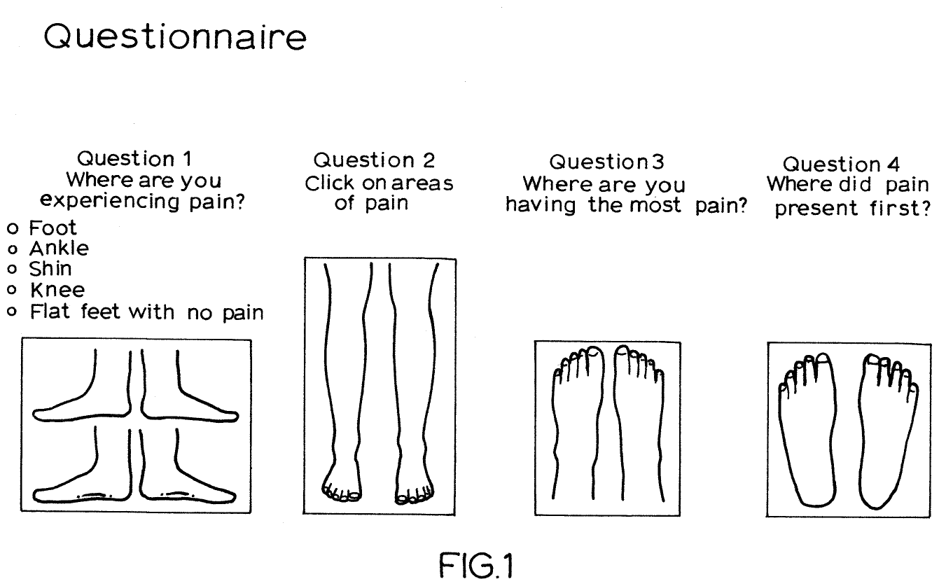

Referring to the drawings, there is shown the operation of the method of providing a foot orthotic to a person according to the preferred embodiment of the invention. The person has presented to them a map of a lower leg that is divided into regions foot, ankle, shin and knee regions. The user selects one or more regions where they are experiencing pain or discomfort and that region/s is presented diagrammatically. The lower part of FIG. 1 shows the various regions where pain may be being experienced for the person to select.

The selections made by the person in FIG. 1 can be presented in any preferred form. In the preferred embodiment, the questionnaire is presented electronically whether shown sequentially or on the same screen. The user firstly selects where they are experiencing pain and then clicks on those area/s.

In the case that a person has multiple areas or regions of pain, a third input is required by the person and this relates to which area has them feeling the most pain. In such cases, the person is then prompted to input where the first area they experienced pain.

It will be appreciated that there are cases where there are no areas of pain but the person is flat footed. In this case, a predetermined orthotic is dispensed.

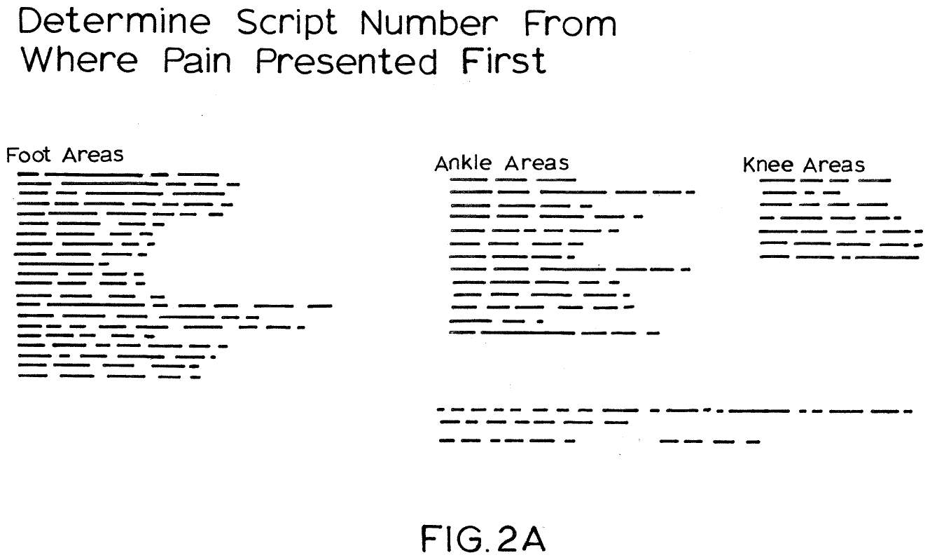

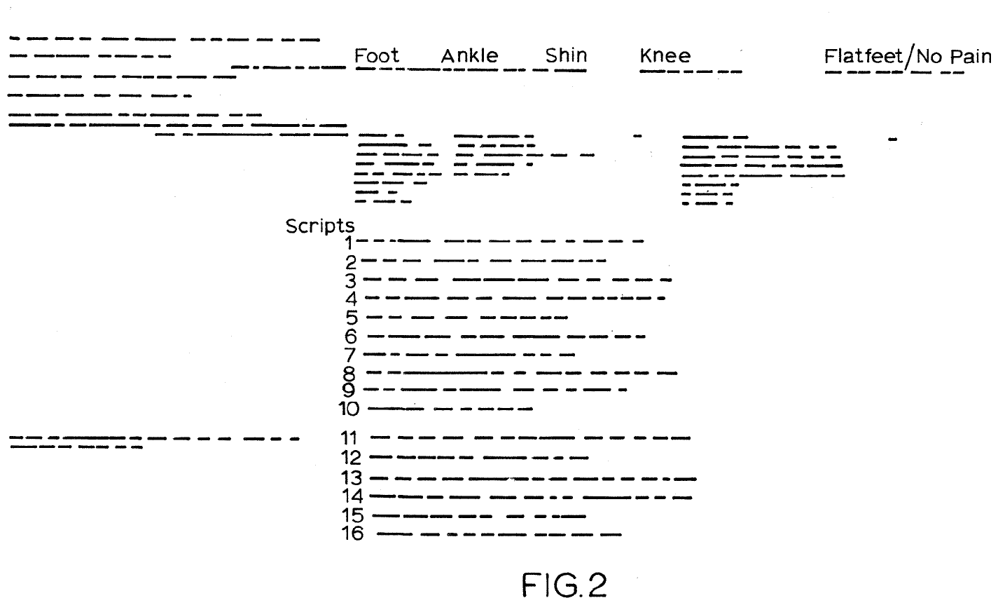

Once the user has selected the region in which they are experiencing pain and have selected the specific area associated with the selected region, one of a set range of predefined foot orthotics is selected to be dispensed to that person. Table 2 is of FIG. 2 showing in tabular form of prescription of orthotic types according to the preferred embodiment of the invention for the person. Production of the orthotics is described further below.

Table of FIG. 2

Where do you experience Pain (Click on the Relevant Box)? Foot Ankle Shin Knee Flatfeet/No pain
Touch Images- Marked areas of pain Plantar, Dorsal, medial, Lateral Posterior, Anterior, Lateral, medial
Skip this stage Anterior, Medial, Lateral Skip this Stage
Click on ALL areas you are experiencing pain- Utilising Images with highlighted areas ------ >?
Of the selected painful areas which area is the most painful (Select one only)?
Please think very carefully as you answer is important- which of the selected pains presented first?
Knee Position- Valgum/Varum/Rectus- Required for Medial and Lateral Joint Line/Compartment Pain,
Achilles Varum/Valgum
Orthotic design Dependent on selected location of pathology Heel MTC (11) Achilles Genu Varum (6) 3
Patella Tendon (1) 3
MCN Entrapment (11) Achilles Genu Valgum (4) Medial Joint Line/Compartment Genu Varum (1)
Plantar Fascia Length (12) Achilles Ensthopathy/Retro Calc Bursitis (9) Medial Joint Line/Compartment
Genu Valgum (3)
Midfoot (Lisfranc) (12) Tib Post tendinopathy (8) Lateral Joint Line/Compartment Genu Varum (1)
Mid Shaft-> MTPJ 2-4 (13) Child Severs (15) Lateral Joint Line/Compartment Genu Valgum (3)
HAV/1st MTPJ (11) ITB Insertion (3)
Styloid (14) PFJ Rectus (2)
2-4 PL MTPJ (13) PFJ Valgum (3)
3-4 Toes (13)
Scripts
1 0* Shell, 4 mm Lateral support, 3 mm Heel Raise, Plantar Fascial Groove, 1st Ray accommodation
2 6* Control, 3 mm Heel Raise, 1st Ray accommodation and Plantar Fascial groove
3 6* rear Control, 3 mm Heel Raise, 3 mm Lateral Support Plantar Fascial groove, 1st Ray
accommodation
4 6* Rear Control, 5 mm heel Raise, PF groove, 1st ray accommodation
5 0* Control, 4 mm Lateral Support, 3 mm Heel Raise, Styloid relief, 1st Ray accommodation, PF groove
6 0* Control, 5 mm heel Raise, 4 mm Lateral Support, 1st Ray accommodation, PF groove
7 6* Control, 5 mm Heel Raise, 4 mm Lateral Support, PF Gove, 1st Ray Accomodation
8 6* Rear Control, 4 mm heel Raise, PF groove, 1st ray accommodation
9 5* Control, 4 mm Lateral Support, 5 mm Heel Raise, PF groove, 1st Ray Acc
10 5* Rear Control, 3 mm Heel Raise, Pl Fascia groove, 4 mm Lateral Support, 1st Ray Acc
11 4* Extended Heel, 4 mm Heel Raise, 1st Ray accommodation, PI Fascia groove
12 5* Control, 4 mm Lateral Support, Pl Fascial groove, 1st Ray accommodation
13 5* Control, Met Dome, 4 mm Lateral Support, Pl Fascial groove, 1st Ray accommodation
14 0* Shell, 6 mm Lateral Support, Styloid accommodation, Pl Fascia groove, 1st Ray accommodation
15 6* Control, 5 mm Heel Raise, Pl Fascis groove, 1st Ray Acc
16 4* extended heel, 1st Ray Accommodation, PF groove, met dome 2-4
Bands Guidelines link above

Table of FIG. 2A
Determine Script Number From
1
Where Pain Presented First

Foot Areas
Flrs1 me1a1arsophalangeal Jo1111 = Scnp111
Second meta1arsopl1alangeall01n1 = Scnp1 13
Third me1a1arsophalangeal101111 = Scnp113
Four1h me1a1arsophalangeal 101n1 = Scrip1 13
Fltth me1a1arsophalangeal101n1 = Scnp114
Second meta1arsal = Scnp1 12
Third me1a1 arsal = Scnp1 12
Founh me1a1arsal = Scrip112
Fltth me1a1arsal = Scnp114
Nav1cular = Scnp1 8
Mid1arsal 101n1s = Scnp1 12
S1yl01d process = Scnp114
An1enor ankle 101n1 = Scnp1 7
Flrs1me1a1arsopl1alangeall0ln1, plan1ar pla1e, sesamo1ds = Scnpt 11
Second plan1ar pla1e, In1erdlgl1al space = Scnp111
Third and 1our1h plan1ar fla1es, In1erdlgl1al space = Scrip111
Fltth plan1ar pla1e = Scnp 14
MerJlal band ot 1he plan1ar tasc1a = Scrip1 12
Cen1ral and La1eral Calcaneus = Scr; p112
Medial 1utlercle calcaneus = Scnp11<.
Medial 1utlercle calcaneus = Scnp112
Ankle Areas
Acl1illes 1endon = Scnp1 6
Achilles Insenion, retrocalcaneal bursa = Scrip 1 6
Peroneal tendon = Scnp1 14
Anterior 1alotibular l Igament = Script 14
Dorsal la1eral mid too1 = Scnp1 14
Styloid process = Script 14
Achilles tendon = Scrip 1 6
Achilles inser1ion, retrocalcaneal bursa = Scrip 1 6
Tiblalis pos1enor tendon = Scnp1 8
Medial 1uoorcle calcaneus = Scnp1 12
Medial band plan1ar tascia = Scnp1 12
N~icular = Scnp1 3
Flrs1 metatarsophalangeal 101nt = Script 11
Knee Areas
La1eral Knee JOIn1 = Scnp11
Pa1ella = Scnp1 1
Medial Knee 101111 = Scnp1 1
Pes ansenne bursa = Scnp1 3
Patella tendon insertion = Script 3
Tltlialls a111, mor 1endon = Scr1p1 8
Anterior border ot sl11n = Scrip1 4
Il Fla1 tee1 no pain and Shin Pain selec1ed a1ques1lon2l11
ques1lonnalre Skip selec1Ing arears of
pa111 and generale scnp1 s1ralght ~vay.
Fla11ee1- no pain: Scnp1 7 Shin Pain: Scnp115

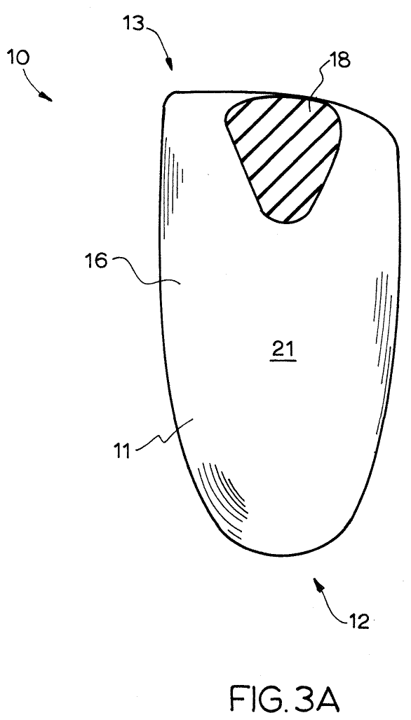

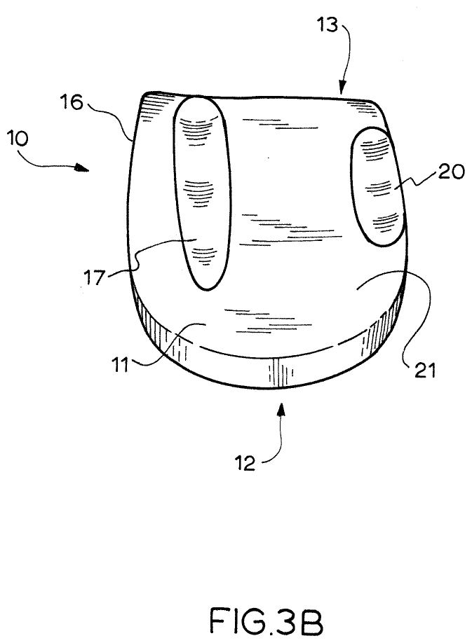

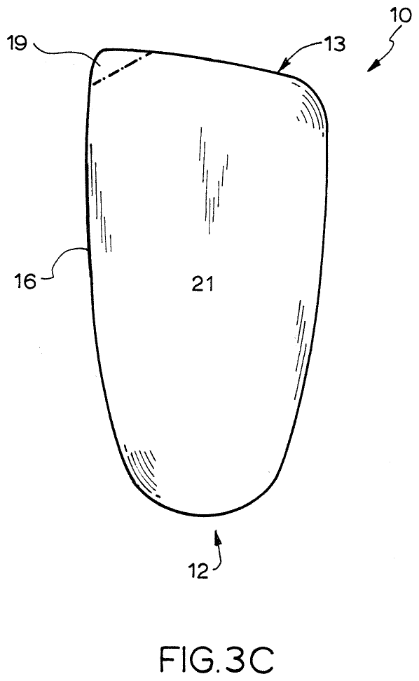

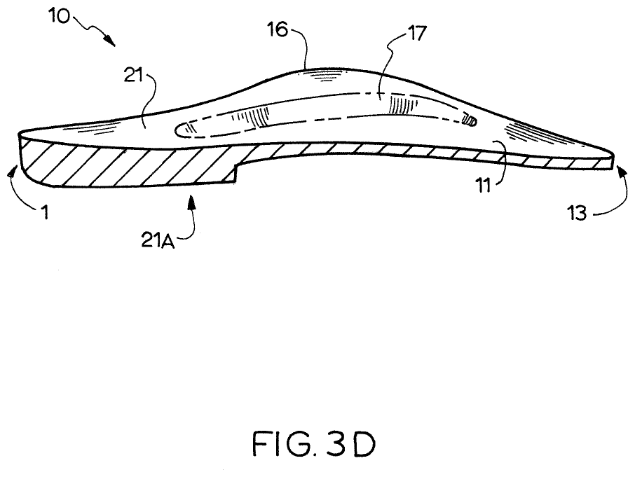

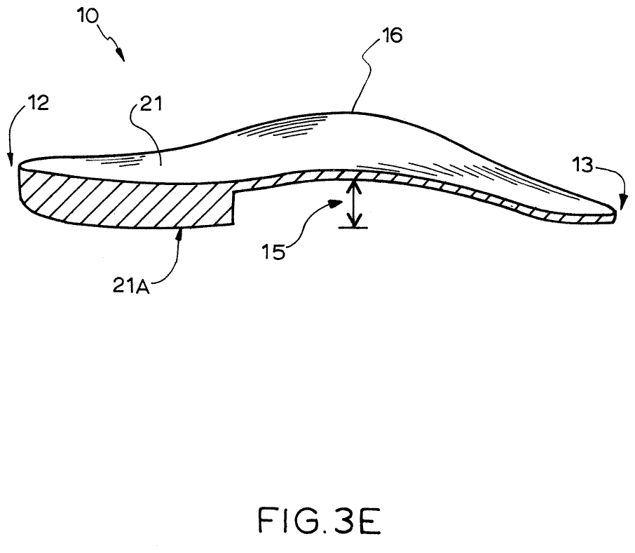

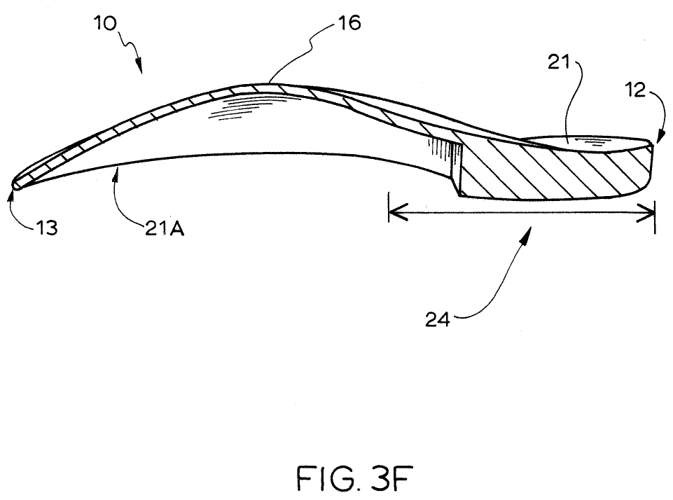

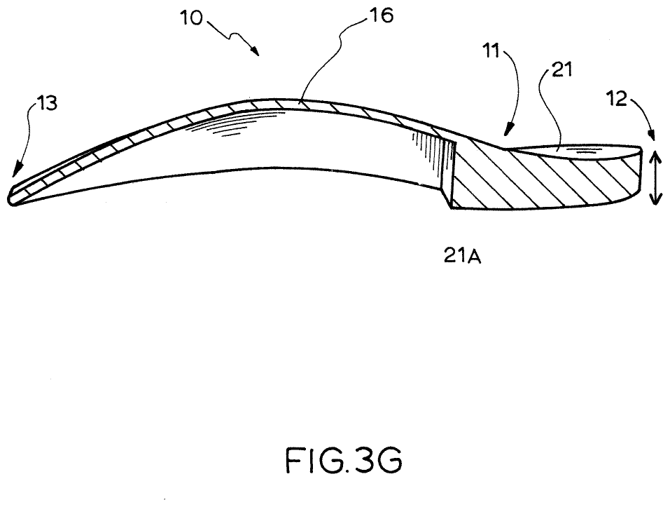

FIG. 3 illustrates the orthotic types to be dispensed subject to the table in FIG. 2 illustrating the orthotic variation locations. The particular orthotics shown in FIG. 3 each has a variation to address the area of pain first experienced by the person. Generally, the orthotics 10 are formed from a three-quarter length orthotic shell 11 which extends between a heel end 12 up to a point 13 corresponding to or about the metatarsophalangeal joints of the person. The orthotic shell 10 is formed to contour to the sole of a foot of the person (not illustrated).

The orthotic shell 10 can have one or more of the variations to its size and/or shape depending on the first area of pain selected by the person. Those variations include 0° to 6° inversion or eversion relative to a midline 14 of the shell 10. A 3 mm to 7 mm high lateral support 15 disposed on an outside of an arch portion 16 of the orthotic shell 10. The heel 12 of the orthotic shell may be raised a predetermined amount and where required a plantar fascia groove 17 having a depth from 0.25 mm to 5 mm is added. Other variations include the addition of a metatarsal dome 18 having a height between 3 to 10 mm with a diameter in the range of 10 to 50 mm. Other modifications required to the shell 10 depending on the first area of pain include a first ray accommodation cut-out 19 between 30° to 90° and the addition of a styloid process accommodation 20 of between 25 mm to 65 mm. The styloid process accommodation 20 is similar to the plantar fascia groove 17 which provides a cutaway from the dorsal accommodation on an outside of the foot orthotic whereas the plantar fascia groove 17 is on a load bearing face 21 of the orthotic 10. On the opposing side of the orthotic load bearing surface 21 is an underside face 21A. Face 21A is adapted to be disposed contiguously with the insole of footwear such as a shoe.

As can be seen in FIG. 1, the predetermined regions of the map of the foot and leg comprise foot areas; ankle areas; & knee areas. Then more specifically, the person selects a specific area of pain from the first selected region. The knee areas include lateral knee joint; patella; medial knee joint; pes anserine or intertendinous bursa; patella tendon; tibialis anterior tendon; anterior border of the tibia; and the tibia.

The ankle areas include Achilles tendon; Achilles insertion; retrocalcaneal bursa; peroneal tendon; anterior talofibula ligament; dorsal lateral midfoot; stylet process; tibialis posterior tendon; medial tubercle calcaneus; medial band plantar fascia; navicular; and first metatarsophalangeal joint.

As shown, the foot areas include first-fifth metatarsophalangeal joints; second-fifth metatarsals; navicular; mid tarsal joints; anterior ankle joint; second to fifth plantar plates; central band of the plantar fascia; central and lateral calcaneus; sesamoid; and plantar plate interdigital spaces.

In the preferred embodiment, the orthotic types include the shell 10 in the form of one of the four general adaptations. These are a control 22 being a mid-foot controlling device having a fulcrum focal about the navicular of the person and adapted to control mid-tarsal joints; a rear control 23 being a rear-foot controlling device having a fulcrum proximal to the talo-navicular joint and adapted to control the sub-talarjoint; an extended heel section 24 adapted to reduce compression at the medial tubucle of the calcaneous and having a fulcrum at the first metatarsal cuneiform joint; and a contour shell 25 adapted to contour to the foot of a person to maximise load distribution and being further adapted to be inverted or everted.

More particularly, the control shell 22, also known as a comfort shell, is a mid-foot controlling device with fulcrum focus at the navicular. It is utilsed to control mid tarsal joints and also in mod pes planus and neutral foot profiles. The rear control 23, also known as a support, is a rearfoot control device-fulcrum proximal to talo navicular joint. This is utilised to control Sub Talar Joint (STJ) in patients that have a medially deviated STJ but is primarly for mod/sig pes planus foot profiles.

In the case of the extended heel 24, also known as a performance shell, provides an elongated heel seat to reduce compression at the medial tubucle of the calcaneus in those patients suffering from plantar fasciitis. This includes a fulcrum at the 1st metatarsal cuneiform joint and is particularly designed for patients suffering from plantar fasciitis.

Contour shell 25 is an accommodative device contouring the patients foot for maximal load distribution. This is utilised primarily in pes caves foot profile feet-fulcrum slightly variable distal to navicular at central apex of the MLA.

Referring to FIG. 3, there is shown the defined foot orthotics 10 prescribed for the person subject to the area of first pain or flat footedness. Specifically, the prescribed orthotics 10 are selected from:
A. 0° contour shell, 4 mm lateral support, 3 mm Heel Raise, Plantar Fascial Grove of depth between 0.75 mm to 1.5 mm, 1st Ray accommodation cutout at 45°.
B. 6° inversion correction comfort shell, 3 mm Heel Raise, Plantar Fascial Grove of depth between 0.75 mm to 1.5 mm, 1st Ray accommodation cutout at 45°.
C. 6° inversion correction support shell, 3 mm Heel Raise, 3 mm Lateral Support, Plantar Fascial Grove of depth between 0.75 mm to 1.5 mm, 1st Ray accommodation cutout at 45°.
D. 6° inversion correction support shell, 5 mm heel Raise, Plantar Fascial Grove of depth between 0.75 mm to 1.5 mm, 1st Ray accommodation cutout at 45°.
E. 0° comfort shell, 4 mm Lateral Support, 3 mm Heel Raise, Styloid relief, Plantar Fascial Grove of depth between 0.75 mm to 1.5 mm, 1st Ray accommodation cutout at 45°.
F. 0° comfort shell, 5 mm heel Raise, 4 mm Lateral Support, Plantar Fascial Grove of depth between 0.75 mm to 1.5 mm, 1st Ray accommodation cutout at 45°.
G. 6° comfort shell, 5 mm Heel Raise, 4 mm Lateral Support, Plantar Fascial Grove of depth between 0.75 mm to 1.5 mm, 1st Ray accommodation cutout at 45°.
H. 6° support shell, 4 mm heel Raise, Plantar Fascial Grove of depth between 0.75 mm to 1.5 mm, 1st Ray accommodation cutout at 45°.
I. 5° comfort shell, 4 mm Lateral Support, 5 mm Heel Raise, Plantar Fascial Grove of depth between 0.75 mm to 1.5 mm, 1st Ray accommodation cutout at 45°.
J. 5° support shell, 3 mm Heel Raise, 4 mm Lateral Support, Plantar Fascial Grove of depth between 0.75 mm to 1.5 mm, 1st Ray accommodation cutout at 45°.
K. 4° inversion correction performance shell, 4 mm Heel Raise, Plantar Fascial Grove of depth between 0.75 mm to 1.5 mm, 1st Ray accommodation cutout at 45°.
L. 5° comfort shell, 4 mm Lateral Support, Plantar Fascial Grove of depth between 0.75 mm to 1.5 mm, 1st Ray accommodation cutout at 45°.
M. 5° comfort shell, metatarsal dome having a height between 3 to 10 mm and a diameter of 10 to 50 mm, 4 mm Lateral Support, Plantar Fascial Grove of depth between 0.75 mm to 1.5 mm, 1st Ray accommodation cutout at 45°.
N. 0° inversion or eversion contour shell, 6 mm Lateral Support, Styloid Accommodation, Plantar Fascial Grove of depth between 0.75 mm to 1.5 mm, 1st Ray accommodation cutout at 45°.
O. 6° comfort shell, 5 mm Heel Raise, Plantar Fascial Grove of depth between 0.75 mm to 1.5 mm, 1st Ray accommodation cutout at 45°.
P. 4° performance shell, Plantar Fascial Grove of depth between 0.75 mm to 1.5 mm, 1st Ray accommodation cutout at 45°, metatarsal dome having a height between 3 to 10 mm and a diameter of 10 to 50 mm.

As can be seen from table 2, the above defined orthotics are dispensed to the person dependent on the area where the person first experienced the pain or discomfort according to:
defined orthotic A in response to first pain at lateral knee joint, patella or medial knee joint, patella tendon, medial or lateral joint line/compartment genu varum;
defined orthotic B in response to first pain at the patellofemoral joint;
defined orthotic C in response to first pain at pes anserine or intertendinous bursa, patellofemoral joint valgum, patella tendon, iliotibial tract, medial or lateral joint line/compartment genu vaglum;
defined orthotic D in response to first pain at Achilles genu valgum, or anterior border of shin;
defined orthotic F in response to first pain at Achilles genu varum, Achilles tendon, Achilles insertion, or retrocalcaneal bursa;
defined orthotic G in response to a person having flat feet and no pain; defined orthotic H in response to first pain at tibialis posterior tendon, tibialis anterior tendon, or navicular;
defined orthotic I in response to first pain at Achilles enthesopathy, or retrocalcaneal bursa;
defined orthotic K in response to first pain at first metatarsophalangeal joint, plantar plate, sesamoid, second plantar plate interdigital space, third and fourth plantar plates interdigital space, heel, medial calcaneal nerve, Hallux abducto valgus, or first metatarsophalangeal joint;
defined orthotic L in response to first pain at plantar fascia length, midfoot lisfranc, second-fourth metatarsal, mid tarsal joints, medial tubercle calcaneus, or medial band plantar fascia.
defined orthotic M in response to first pain at second-fourth metatarsophalangeal joint midshaft or PL, or third or fourth toes.
defined orthotic N in response to first pain at styloid, fifth metatarsophalangeal joint, fifth plantar plate, peroneal tendon, anterior talofibula ligament, dorsal lateral midfoot, and styloid process.
defined orthotic O in response to first pain at the tibia/shin.

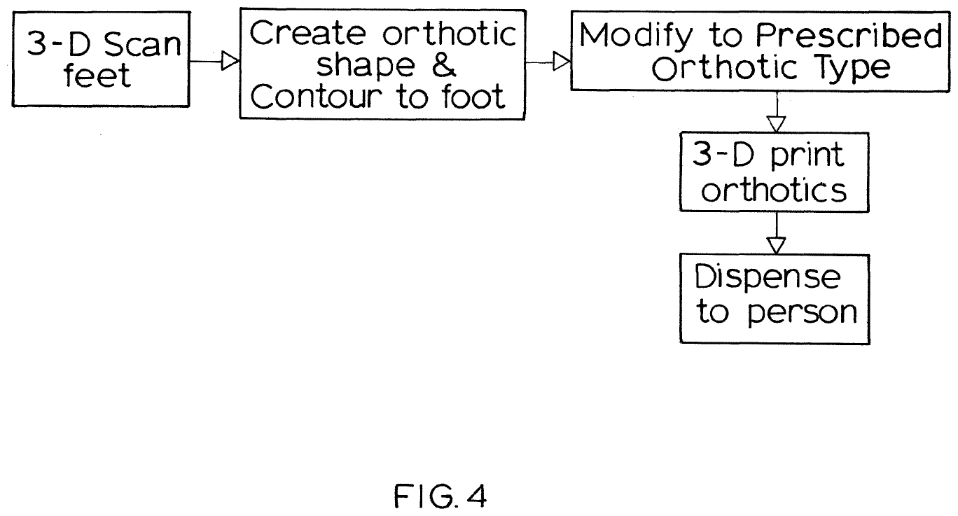

FIG. 4 shows a schematic process diagram for the production of a defined orthotic A-P in the preferred embodiment. Once prescribed, each sole of foot of the person is scanned or imaged most preferably with the foot in the neutral position where the foot is neither pronated nor supinated. It is noted both feet are scanned and a foot not exhibiting pain or flat footedness is prescribed an orthotic shell 10 that merely conforms to the shape of that foot.

The feet can be imaged in any preferred manner such as 3-D laser scanning or rendering and this can be performed by the person if desirable or convenient. For example cameras or smartphones having time of flight 3-D imaging, or LIDAR systems can produce 3-D images of the foot, or other means such as impression boxes or pressure plates for the feet can be employed. Once scanned the prescribed orthotic as above is produced with the defined variations by any preferred means including 3-D printing or by moulding or forming a cast. For example, the depth of the plantar facia groove 16 depth or the height of the metatarsal dome height amongst the others variations can be scaled from the scanned image/s. When 3-D printed for example, the orthotics 10 can be formed from scratch or from a blank (not illustrated) that is modified by printed and/or machining.

Set out in the table below are the results of a research trial of people who have been experiencing areas of pain or discomfort as selectable from the questionnaire of FIG. 1 described above. The area/s where pain was first experienced were noted and the corresponding defined orthotic selected as described above.

| Region | Area of First Pain | Prescribed | Number | Result/Comment |
|---|---|---|---|---|
| — | — | Orthotic | of people | — |
| — | — | A-P | — | — |
| Knee | lateral knee joint | C | 10 | Associated genu valgum knee |
| | | | | position, application of orthotic |
| | | | | with a 6 degree rearfoot posting to |
| | | | | provide a medial force to the sub |
| | | | | talar joint, and good MLA contour |
| | | | | allowed for a decompression at the |
| | | | | lateral knee joint. |
| | patella | A | 8 | The prescribed orthotic provided a |
| | | | | valgus moment through the midfoot, |
| | | | | assisting in ensuring 1^{st} ray |
| | | | | propulsion and reduced external |
| | | | | rotation moment at the knee. |
| | medial knee joint | A | 15 | This defined orthotic provided |
| | | | | increased lateral arch profile and |
| | | | | promoted 1^{st} ray propulsion |
| | | | | leading to a decompression at the |
| | | | | medial joint margin. It was |
| | | | | incredibly effective at resolving |
| | | | | discomfort. |
| | pes anserine or | | | |
| | intertendinous bursa | | | |
| | patella tendon | B or C | 20 | Pathology associated with mal- |
| | | Dependent | | tracking of the patella tendon in the |
| | | on varum | | femoral condyle. The two orthotic |
| | | or valgum | | designs applied 6 degree inversion |
| | | angle of | | moment, Orthotic (B) acting at the |
| | | knee | | mid tarsal joint and (C) acting at |
| | | | | the sub talar joint as required. |
| | tibialis anterior | H | 15 | Function Tibialis anterior tendon |
| | tendon | | | inverted and dorsiflexed the foot. |
| | | | | The application of the orthotic |
| | | | | device applied an inversion moment |
| | | | | at the Sub Talar Joint and the |
| | | | | pitched 4 mm heel raise engaged |
| | | | | the Tibialis posterior and gastric- |
| | | | | soleus. |
| | anterior border of | B | 10 | Reduction of tractional load at the |
| | the tibia | | | periosteal junction of the anterior |
| | | | | border of the tibia. The application |
| | | | | of the orthotic device provided an |
| | | | | inversion moment at the sub talar |
| | | | | joint and mid tarsal joint, the |
| | | | | resultant reduction in soft tissue |
| | | | | tractional load and resolving pain |
| | | | | presentation. |
| | Achilles tendon | D and F | 25 | Application of a 5 mm heel raise, |
| | | dependent | | with supported load distribution |
| | | on Varum | | resulted in significant pain |
| | | or Valgum | | reduction |
| | | angle | | |
| | Achilles insertion | I | 15 | 5 mm heel raise, with increased |
| | | | | lateral arch support facilitates 1^{st} |
| | | | | ray propulsion and reduced |
| | | | | tractional load at the achillies |
| | | | | insertion. |
| | retrocalcaneal | I | 15 | As above- increased plantar flexed |
| | bursa | | | moment reduced compression at |
| | | | | the retro calc bursa through single |
| | | | | leg stance phase. |
| | peroneal tendon | N | 10 | Increased lateral arch profile |
| | | | | resulted in an increased transition |
| | | | | of lateral load forces towards the |
| | | | | medial plane providing a reduction |
| | | | | of peroneal tendon loading forces. |
| | anterior talofibula | L | 15 | Application of 5 degree rearfoot |
| | ligament | | | inversion coupled with 4 mm lateral |
| | | | | arch support and 1^{st} ray assisted in |
| | | | | stabilising the lateral ankle complex |
| | dorsal lateral | M | 8 | Utilised 5 degree of rearfoot |
| | midfoot | | | inversion coupled with 4 mm lateral |
| | | | | arch support and a met dome. This |
| | | | | provided lateral arch support and |
| | | | | custom contour ensuring a reduction |
| | | | | in dorsal joint compression and a |
| | | | | shift of forces towards the medial |
| | | | | axis. |
| | styloid process | N | 25 | Associated with lateral column |
| | | | | overload leading to increased |
| | | | | traction at the styloid process from |
| | | | | the peroneal brevis tendon. Defined |
| | | | | orthotic N applied increased lateral |
| | | | | column support to shift the load |
| | | | | toward the medial axis as well as a |
| | | | | dorsal accommodation to assist in |
| | | | | offload at the styloid site. |
| | tibialis posterior | H | 50 | Applied a medial inversion force to |
| | tendon | | | the sub talar joint and addition of |
| | | | | 4 mm heel raise to increase gastroc |
| | | | | soleal activation. |
| | medial tubercle | K | 100 | Applied an extended heel seat |
| | calcaneus | | | meaning reduction of compressive |
| | | | | force at the MTC. This coupled |
| | | | | with 4 degrees of rearfoot inversion |
| | | | | and a large plantar fascial grove |
| | | | | significantly reduced the tractional |
| | | | | load of the plantar fascia at the MTC. |
| | medial band plantar | K | 80 | As above |
| | fascia | | | |
| | navicular | L | 30 | Mid point of MLA overlaying |
| | | | | navicular, applied 5 degree rearfoot |
| | | | | inversion with coupled lateral |
| | | | | support to stabilised the calcaneo |
| | | | | cuboid joint to promote anatomical |
| | | | | stability. |
| | first | K | 50 | Utilised extended heel seat device |
| | metatarsophalangeal | | | means so that the MLA apex was |
| | joint. | | | distal at the 1^{st} metatarsal cuneiform |
| | | | | joint providing a supported plantar |
| | | | | flexion moment of the 1st MTPJ to |
| | | | | allow for increased Dordi flexion |
| | | | | motion through propulsion phase of |
| | | | | gait cycle. |
| Foot | | | | |
| | first-fifth | B | 20 | Mid foot controlling device utilised |
| | metatarsophalangeal | | | good contour with 6 degree of |
| | joints | | | rearfoot. Even distribution of load |
| | | | | to redirect to midfoot away from |
| | | | | forefoot. |
| | second-fifth | B | 25 | As above |
| | metatarsal | | | |
| | navicular; mid | L | 50 | See above previously addressed |
| | tarsal joints | | | |
| | anterior ankle | G | 30 | Application of 5 mm heel raise and |
| | joint | | | lateral support to stabilise calcaneo |
| | | | | cuboid joint. This promoted an |
| | | | | increased plantar flexed position to |
| | | | | reduce anterior ankle compression |
| | | | | through mid stance cycle of gait. |
| | second to fifth | M | 20 | Promotion of mid foot control, with |
| | plantar plates | | | added met dome applying metatarsal |
| | | | | dorsiflexion and plantar plate offload. |
| | central band of the | K | 100 | Previously addressed above Medial |
| | plantar fascia | | | band of plantar fascia |
| | central and lateral | K | 50 | As discussed above-extended heel |
| | calcaneus | | | seat allowed for greater calcaneal |
| | | | | load distribution. |
| | sesamoid | K | 20 | Promoted greater proximal load |
| | | | | distribution to 1^{st} metatarsal shaft - |
| | | | | reducing load at plantar 1^{st} MTPJ. |
| | plantar plate | M | 25 | Same as per second to fifth plantar |
| | interdigital spaces. | | | plates above. |
| Flat | No pain region. | A | 30 | Application of accommodative device, |
| Feet | | | | increased lateral arch support to assist |
| | | | | in calcaneal cuboid joint stability. |

As can be seen, the preferred embodiments of the invention provide a method of producing and dispensing a foot orthotic to a person without the need to engage a specialist practitioner. It was found that the orthotics achieved the desired result so far as the people involved in the trial reported reduced pain &/or discomfort after use of one of the defined orthotic types. The orthotics produced in the trial were not limited to any particular fabrication techniques and were formed from either 3-D scanning or impression of the trial participant's feet and so anatomically conformed to the foot of the person with prescribed modifications.

The foregoing describes only one embodiment of the present invention and modifications, obvious to those skilled in the art, can be made thereto without departing from the scope of the present invention.

The term “comprising” (and its grammatical variations) as used herein is used in the inclusive sense of “including” or “having” and not in the exclusive sense of “consisting only of”.

1. A method of providing a foot orthotic to a person in need thereof, the method including the steps of:
presenting a map of a foot and leg divided into predetermined regions to the person; receiving a selection from the person of one or more regions from the map corresponding to one or more locations where the person is experiencing pain or discomfort; defining a plurality of foot orthotic types, each foot orthotic type adapted to address pain or discomfort in the one or more regions where the person is experiencing pain or discomfort;
dispensing an orthotic from the plurality of orthotic types such that the defined orthotic is adapted to address pain or discomfort in the region where the person first experienced the pain or discomfort.

2. The method according to claim 1 wherein the defined plurality of foot orthotic types are formed from a ¾ length orthotic shell adapted to extend between the heel and to or adjacent to the metatarsophalangeal joints, the orthotic shell adapted to contour to the sole of a foot of the person.

3. A method according to claim 2 wherein the orthotic types comprise having one or more of the variations consisting of: 0° to 6° inversion or eversion; 3 mm to 7 mm lateral support; 2 mm to 6 mm heel raise; a plantar fascia groove having a depth from 0.25 mm to 5 mm; metatarsal dome having a height between 3 to 10 mm and a diameter of 10 to 50 mm; first ray accommodation cut-out between 30° to 90°; and styloid process accommodation of between 25 mm to 65 mm.

4. The method according to claim 1 wherein the predetermined regions of the map of the foot and leg comprise foot areas; ankle areas; and knee areas.

5. The method according to claim 4 wherein the knee areas include lateral knee joint; patella; medial knee joint; pes anserine or intertendinous bursa; patella tendon; tibialis anterior tendon; anterior border of the tibia; and the tibia.

6. The method according to claim 5 wherein the ankle areas include Achilles tendon; Achilles insertion; retrocalcaneal bursa; peroneal tendon; anterior talofibula ligament;
dorsal lateral midfoot; stylet process; tibialis posterior tendon; medial tubercle calcaneus; medial band plantar fascia; navicular; and first metatarsophalangeal joint.

7. The method according to claim 4 wherein the foot areas include first-fifth metatarsophalangeal joints; second-fifth metatarsals; navicular; mid tarsal joints; anterior ankle joint; second to fifth plantar plates; central band of the plantar fascia; central and lateral calcaneus; sesamoid; and plantar plate interdigital spaces.

8. The method according to claim 4 wherein a predetermined region on the map of the leg and foot includes a flat feet with no pain region.

9. The method according to claim 3 wherein the orthotic types include a shell in the form of:
a control being a mid-foot controlling device having a fulcrum focal about the navicular and adapted to control mid-tarsal joints;
a rear control being a rear-foot controlling device having a fulcrum proximal to the talo navicular joint and adapted to control the sub-talar joint;
an extended heel section adapted to reduce compression at the medial tubucle of the calcaneous and having a fulcrum at the first metatarsal cuneiform joint;
a contour shell adapted to contour to the foot of a patient to maximise load distribution and being further adapted to be inverted or everted.

10. The method according to claim 9 wherein the defined foot orthotics are selected from the group consisting of an orthotic shell having:
A. 0° contour shell, 4 mm lateral support, 3 mm Heel Raise, Plantar Fascial Grove of depth between 0.75 mm to 1.5 mm, 1st Ray accommodation cutout at 45°,
B. 6° inversion correction comfort shell, 3 mm Heel Raise, Plantar Fascial Grove of depth between 0.75 mm to 1.5 mm, 1st Ray accommodation cutout at 45°,
C. 6° inversion correction support shell, 3 mm Heel Raise, 3 mm Lateral Support, Plantar Fascial Grove of depth between 0.75 mm to 1.5 mm, 1st Ray accommodation cutout at 45°,
D. 6° inversion correction support shell, 5 mm heel Raise, Plantar Fascial Grove of depth between 0.75 mm to 1.5 mm, 1st Ray accommodation cutout at 45°,
E. 0° comfort shell, 4 mm Lateral Support, 3 mm Heel Raise, Styloid relief, Plantar Fascial Grove of depth between 0.75 mm to 1.5 mm, 1st Ray accommodation cutout at 45°,
F. 0° comfort shell, 5 mm heel Raise, 4 mm Lateral Support, Plantar Fascial Grove of depth between 0.75 mm to 1.5 mm, 1st Ray accommodation cutout at 45°,
G. 6° comfort shell, 5 mm Heel Raise, 4 mm Lateral Support, Plantar Fascial Grove of depth between 0.75 mm to 1.5 mm, 1st Ray accommodation cutout at 45°,
H. 6° support shell, 4 mm heel Raise, Plantar Fascial Grove of depth between 0.75 mm to 1.5 mm, 1st Ray accommodation cutout at 45°,
I. 5° comfort shell, 4 mm Lateral Support, 5 mm Heel Raise, Plantar Fascial Grove of depth between 0.75 mm to 1.5 mm, 1st Ray accommodation cutout at 45°,
J. 5° support shell, 3 mm Heel Raise, 4 mm Lateral Support, Plantar Fascial Grove of depth between 0.75 mm to 1.5 mm, 1st Ray accommodation cutout at 45°,
K. 4° inversion correction performance shell, 4 mm Heel Raise, Plantar Fascial Grove of depth between 0.75 mm to 1.5 mm, 1st Ray accommodation cutout at 45°,
L. 5° comfort shell, 4 mm Lateral Support, Plantar Fascial Grove of depth between 0.75 mm to 1.5 mm, 1st Ray accommodation cutout at 45°,
M. 5° comfort shell, metatarsal dome having a height between 3 to 10 mm and a diameter of 10 to 50 mm, 4 mm Lateral Support, Plantar Fascial Grove of depth between 0.75 mm to 1.5 mm, 1st Ray accommodation cutout at 45°,
N. 0° inversion or eversion contour shell, 6 mm Lateral Support, Styloid Accommodation, Plantar Fascial Grove of depth between 0.75 mm to 1.5 mm, 1st Ray accommodation cutout at 45°,
O. 6° comfort shell, 5 mm Heel Raise, Plantar Fascial Grove of depth between 0.75 mm to 1.5 mm, 1st Ray accommodation cutout at 45°,
P. 4° performance shell, Plantar Fascial Grove of depth between 0.75 mm to 1.5 mm, 1st Ray accommodation cutout at 45°, metatarsal dome having a height between 3 to 10 mm and a diameter of 10 to 50 mm.

11. The method according to claim 10 wherein the defined foot orthotics are dispensed to the person dependent on the region where the person first experienced the pain or discomfort according to:
defined orthotic A in response to first pain at lateral knee joint, patella or medial knee joint, patella tendon, medial or lateral joint line/compartment genu varum;
defined orthotic B in response to first pain at the patellofemoral joint;
defined orthotic C in response to first pain at pes anserine or intertendinous bursa, patellofemoral joint valgum, patella tendon, iliotibial tract, medial or lateral joint line/compartment genu vaglum;
defined orthotic D in response to first pain at Achilles genu valgum, or anterior border of shin;
defined orthotic F in response to first pain at Achilles genu varum, Achilles tendon, Achilles insertion, or retrocalcaneal bursa;
defined orthotic G in response to a person having flat feet and no pain;
defined orthotic H in response to first pain at tibialis posterior tendon, tibialis anterior tendon, or navicular;
defined orthotic I in response to first pain at Achilles enthesopathy, or retrocalcaneal bursa;
defined orthotic K in response to first pain at first metatarsophalangeal joint, plantar plate, sesamoid, second plantar plate interdigital space, third and fourth plantar plates interdigital space, heel MTC <please note I am unsure if MTC is medial tubercle calcaneus as minimum toe clearance does not indicate the location of pain>, medial calcaneal nerve, Hallux abducto valgus, or first metatarsophalangeal joint;
defined orthotic L in response to first pain at plantar fascia length, midfoot lisfranc, second-fourth metatarsal, mid tarsal joints, medial tubercle calcaneus, or medial band plantar fascia.
defined orthotic M in response to first pain at second-fourth metatarsophalangeal joint midshaft or PL, or third or fourth toes.
defined orthotic N in response to first pain at styloid, fifth metatarsophalangeal joint, fifth plantar plate, peroneal tendon, anterior talofibula ligament, dorsal lateral midfoot, and styloid process.
defined orthotic O in response to first pain at the tibia/shin;
defined orthotic P in response to first pain at <1 cannot see a first area of pain corresponding to script 16>.

12. The method according to claim 10 further including the step of measuring or 3-D scanning the person's foot and 3-D printing a defined foot orthotic being dimensioned to correspond to the person's foot.

13. The method according to claim 12 wherein the defined foot orthotic is 3D printed on a blank foot orthotic.

14. The method according to claim 12 or 13 wherein the foot of the person is electronically scanned by the person or by a third party.

15. The method according to claim 2 wherein the predetermined regions of the map of the foot and leg comprise foot areas; ankle areas; and knee areas.

16. The method according to claim 3 wherein the predetermined regions of the map of the foot and leg comprise foot areas; ankle areas; and knee areas.

17. The method according to claim 13 wherein the foot of the person is electronically scanned by the person or by a third party.
